(19) United States
(12) Patent Application Publication
(10) Pub. No.: US 2018/0235936 A1
(43) Pub. Date: Aug. 23, 2018

(54) CANCER TREATMENT METHODS

(71) Applicant: University of Notre Dame du Lac, South Bend, IN (US)

(72) Inventor: Katherine Richards, South Bend, IN (US)

(21) Appl. No.: 15/898,545

(22) Filed: Feb. 17, 2018

Related U.S. Application Data
(60) Provisional application No. 62/460,531, filed on Feb. 17, 2017.

(51) Int. Cl. A61K 31/4178; A61P 35/00; A61K 45/06

(52) U.S. Cl. CPC A61K 31/4178; A61K 45/06; A61P 35/00

(57) ABSTRACT

Methods for treating cancer include administering and/or receiving, at a patient in need thereof, a therapeutic dose of an inhibitor of extracellular vesicle signaling. The methods can additionally include co-administering and/or additionally receiving, at the patient in need thereof, a therapeutic dose or regimen of chemotherapy. In some instances, the cancer includes solid tumor carcinomas such as, but not limited to, colorectal cancer, pancreatic cancer, breast cancer, lung cancer, and prostate cancer. The disclosed treatment methods can act to suppress one or more of tumor growth, resistance to drug therapy, evasion of apoptosis, and/or metastasis.

### CROSS-REFERENCE TO RELATED APPLICATIONS

This application claims priority to and the benefit of U.S. Provisional Patent Application Ser. No. 62/460,531, filed Feb. 17, 2017 and titled “EXOSOMES IN CARCINOMA TREATMENT.” The foregoing is incorporated herein by reference in its entirety.

### BACKGROUND

### Technical Field

This disclosure generally relates to cancer treatment methods. More specifically, the present disclosure relates to cancer treatment methods using chemical and/or molecular inhibitors of extracellular vesicle signaling.

### Related Technology

Pancreatic ductal adenocarcinoma (PDAC) has a dismal 5-year survival rate of less than 7%. PDAC is currently the fourth leading cause of cancer-related deaths in the United States and is predicted to become the second leading cause of cancer-related deaths by 2030. The poor prognosis of the disease is associated with late detection, aggressive tumor biology, and poor response to available therapies. Gemcitabine, a nucleoside analog, is the standard chemotherapeutic agent for adjuvant therapy of resectable PDAC and a commonly used agent in other treatment settings, including neoadjuvant treatment of borderline resectable PDAC and palliative treatment of metastatic PDAC. Despite gemcitabine being one of the most commonly used, single chemotherapeutic agents in pancreatic cancer, response rates are poor. The vast majority of patients (e.g., 74% of patients in an observed cohort) receiving adjuvant gemcitabine eventually show tumor recurrence. This dismal prognosis shows an urgent need for greater understanding of the complete biology of PDAC in order to develop effective therapeutic strategies.

During tumorigenesis stromal tissue, comprising fibroblasts, increases in volume surrounding sites of hyperplasia, metaplasia, dysplasia, inflammation, and neoplasia. Higher than normal number and volume of fibroblast cells surround early precursor cellular lesions known as pancreatic intraepithelial neoplasias (PanlNs) and acinar to ductal metaplasias (ADMs), both of which are thought to be the original sites of cancer cell development and tumor formation. While tumorigenesis persists, more fibroblasts surround the cancer tissue, and pancreatic ductal adenocarcinoma (PDAC) tumors end up largely consisting of stromal tissue and fibroblasts. Even though approximately 90% of PDAC tumor volume consists of stromal tissue, current therapies focus predominantly on targeting the proliferation of the rapidly growing epithelial cancer cells.

Overall, gemcitabine, the standard of care chemotherapeutic, does not significantly increase overall survival time of pancreatic cancer patients. Moreover, gemcitabine has already proven to be a paradoxical drug as it not only promotes expression of the Snai1 transcription factor (i.e., protein expression from the SNAI1 gene), but also triggers NF-κB activation, CXCR4 expression, induction of reactive oxygen species, upregulation of cancer-stem cell markers and AKT activity. Together, these undesired side effects lead to increased chemoresistance and cell motility.

Accordingly, there are a number of disadvantages with current cancer treatment methods that can be addressed.

### BRIEF SUMMARY

Embodiments of the present disclosure solve one or more of the foregoing or other problems in the art with cancer treatment therapies. In particular, one or more implementations can include a method for treating cancer that includes receiving, at a subject in need thereof, a therapeutic dose of an inhibitor of extracellular vesicle signaling. The method can additionally include receiving, at the subject in need thereof, a therapeutic dose or regimen of chemotherapy.

Methods of the present disclosure can also include administering a therapeutic dose of an inhibitor of extracellular vesicle signaling to a subject in need thereof. Additionally, methods can include co-administering a therapeutic dose or regimen of chemotherapy to the subject in need thereof.

In some embodiments, the inhibitor of extracellular vesicle signaling includes GW4869.

In some embodiments, treating cancer comprises treating one or more of colorectal cancer, pancreatic cancer, breast cancer, lung cancer, or prostate cancer. In an exemplary embodiment, treating cancer comprises treating pancreatic cancer.

Accordingly, methods for treating cancer are disclosed, the results of which can include, in one or more embodiments, any of a suppression of chemoresistance, tumor growth, metastasis, or other therapeutic benefit.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which AsPC1, L3.6, and PANC1 are all pancreatic cancer epithelial cell lines and:

FIGS. 1A-D include microscopy images and graphs illustrating the innately chemoresistant nature of pancreatic fibroblasts. (FIG. 1A) Immunofluorescence stain for aSMA and vimentin of pancreatic cancer-associated fibroblasts (CAF1) and wild-type (WT) fibroblasts. (FIG. 1B) Cells were treated with 111M gemcitabine for 2-6 days and live and dead cells were counted to obtain percent cell survival. (FIG. 1C) Cells were treated with 111M gemcitabine for 2 days or left untreated and total cells were counted to obtain percentage of proliferation retention during GEM treatment. (FIG. 1D) Percent cell survival of CAFs (CAF2) and epithelial cells (PANC1) with similar proliferation retention rate over 6 days 111M gemcitabine treatment. **p-value<0.01.

FIG. 2 illustrates a sequence alignment of patient-derived pancreatic cancer-associated fibroblasts (CAFs), demonstrating a KRAS wild type phenotype. CAF lines CAF1 (SEQ ID NO: 1) and CAF2 (SEQ ID NO: 2) were sequenced to determine if KRAS mutations were present in exon 2 at codon 12 and 13 (black outlined area). Both cell lines were identified as KRAS wild type (SEQ ID NO: 3).

FIGS. 3A-D demonstrate pancreatic CAF1-conditioned media increasing proliferation and survival of epithelial cells. (FIG. 3A) L3.6 cells were grown in CAF1-conditioned media or L3.6-conditioned media for 8 days and total cells were counted. (FIG. 3B) Cell proliferation assay (MTT assay) was performed after 8 days L3.6 cell growth in conditioned media. (FIG. 3C) L3.6 cells were grown in cell-conditioned media for 6 days then treated with 100 nM gemcitabine for 3 days, and live cells were counted. (FIG. 3D) Cell proliferation assay (MTT) was performed after 6 days L3.6 cell growth in conditioned media and 3 days of 100 nM gemcitabine treatment. **p-value<0.01.

FIGS. 4A-D demonstrate gemcitabine increasing CAF exosome secretion. (FIG. 4A) CAF1s were left untreated (NT) or treated with 111M gem (GT) for 4 days. Exosomes were isolated from conditioned cell media, and protein lysates were used to perform a western blot for CD81 and beta-actin (left). Isolated exosomes were examined for size and structure via transmission electron microscopy (right). (FIG. 4B) CAF1 cells transduced with a GFP-CD63 lentivirus (GFP-CD63-CAF1s) were treated with luM gemcitabine (GT) or left untreated (NT) and fluorescence was analyzed via microscopy. (FIG. 4C) Total corrected cell fluorescence of GFP-CD63-CAF1 cells was quantified using ImageJ. (FIG. 4D) Cells were treated with 11.1M gemcitabine (Fibroblasts and PANC1), 10 nM gemcitabine (L3.6), or left untreated for 4 days (NT), and exosomes were collected and quantified. Scale bar, 200 Em. *pvalue<0.05; **p-value<0.01.

FIG. 5 illustrates a graph of fold change in exosome secretion per cell. CAF1 cells were plated at 500,000 cells/flask and left untreated (control), treated with 111M gemcitabine, or 10 nM Nab-Paclitaxel for 4 days. Live cells were counted. Exosomes were isolated from conditioned-media and quantified. *p-value<0.05;**p-value<0.01.

FIGS. 6A-E demonstrate GT-CAF exosomes increasing cell number and survival of epithelial cells. (FIG. 6A) L3.6 cells were treated with GFP-CD63-CAF1 conditioned media for 48 hours and exosome uptake was visualized. (FIG. 6B) L3.6 cells were treated with L3.6, GT-PANC1, or GT-CAF1 exosomes for 6 days and total cells were counted. (FIG. 6C) L3.6 cells were treated with L3.6, GT-PANC1 or GT-CAF1 exosomes for 6 days and 111M GEM for 3 days, and live cells were counted. (FIG. 6D) PANC1 cells were treated with PANC1 or GT-CAF1 exosomes for 6 days. AsPC1 cells were treated with AsPC1 or GT-CAF1 exosomes for 6 days. Total cells were counted. (FIG. 6E) PANC1 cells were treated with PANC1 or GT-CAF1 exosomes for 6 days, and AsPC1 cells were treated with AsPC1 or GTCAF1 exosomes for 6 days. All cells were then treated with 111M GEM for 3 days, and live cells were counted. *p-value<0.05; **p-value<0.01.

FIGS. 7A-B illustrates graphs of AsPC1 cells treated directly with AsPC1 exosomes (AsPC1), untreated CAF1 exosomes (CAF-NT), and GEM-treated CAF1 exosomes (CAF-GT) for 6 days. Total cells were counted (FIG. 7A) or were subsequently treated with 111M gemcitabine for 3 days and live cells were counted (FIG. 7B). *p-value<0.05;**p-value <0.01; NS (not significant).

FIGS. 8A-E: demonstrate inhibition of CAF exosome signaling suppressing chemoresistance. (FIG. 8A) AsPC1 cells were grown for 5 days in AsPC1-conditioned media (AsPC1/AsPC1), CAF1-conditioned media (CAF1/AsPC1) or CAF1-conditioned media depleted of exosomes (CAF1-ED/AsPC1) and then treated with 111M GEM for 3 days and live cells were counted. (FIG. 8B) CAF1s were treated with 20 gm GW4869 or DMSO along with 111M gemcitabine or PBS for 3 days. Exosomes in media were collected, dyed with CFSE, and quantified. (FIG. 8C) AsPC1 cells were co-cultured with AsPC1 cells, CAFs, GW4869-treated CAF1s, DMEM alone (Blank/AsPC1), or GW4869 in DMEM (Blank+GW4869) for 6 days then treated during co-culture with 111M gemcitabine for 3 days. Live co-cultured AsPC1 cells at the bottom of the plate were counted. (FIG. 8D) Snai1 expression was measured via RT-PCR in AsPC1 cells co-cultured with untreated CAFs (CAF-NT/AsPC1) or GW4869-treated CAFs (CAF-GW/AsPC1). (FIG. 8E) L3.6 cells were cultured in CAF1-conditioned (CAF1/L3.6) media or CAF1-conditioned media depleted of exosomes (CAF1-ED/L3.6). miR146a expression was measured via RT-PCR. **p-value<0.01.

FIGS. 9A-E demonstrate graphs of epithelial cells co-cultured with DMSO-treated epithelial cells, DMSO treated CAFs, or GW4869-treated CAFs (20 μM) plated on 0.4 μm pore inserts for three days. The bottom co-cultured epithelial cells were then treated with gemcitabine for three days during co-culture. Live co-cultured epithelial cells at the bottom of the plate were quantified using an automated cell counter. *p-value<0.05; **p-value <0.01. NS (not significant).

FIGS. 10A-B illustrate graphs of NOD/SCID mice that were subcutaneously implanted with one million AsPC1 cells and 200,000 CAF1 cells. Two weeks post implantation mice were treated intraperitoneally with DMSO+PBS, DMSO+gemcitabine (GEM), or PBS+GW4869 twice weekly for two weeks. (FIG. 10A) Tumor growth over the course of the ten-day post drug treatment. (FIG. 10B) Change in tumor volume from first day of drug treatment to 10 days post initial drug treatment. *p-value<0.05. NS (not significant).

FIGS. 11A-E demonstrate pancreatic fibroblasts upregulating and secreting miR-146a and Snai1 during gemcitabine treatment. (FIG. 11A) RT-PCR. miR-146a and Snai1 levels were altered in CAF1s during 1 μM GEM treatment (GT) (3 days) compared to untreated control (NT). (FIG. 11B) RT-PCR. CAF1s were treated with Snai1-siRNA, and Snai1 and miR-146a expression was measured compared to negative siRNA control treated CAFs. (FIG. 11C) Exosomes from untreated and 1 μM GEM-treated CAF1s were isolated and Snai1 mRNA and miR-146a within CAF1 exosomes was quantified via RT-PCR using relative Ct values. (FIG. 11D-E) L3.6 cells were treated with GT-CAF1 exosomes for 6 days (GTCAF1/L3.6) or left untreated (L3.6 control). AsPC1 cells were treated with GT-CAF1 exosomes for 6 days (GT-CAF1/AsPC1) or left untreated (AsPC1 control). Snai1 (FIG. 11D) and miR-146a (FIG. 11E) levels were quantified in recipient cells via RT-PCR. *p-value<0.05; **p-value<0.01.

FIG. 12 illustrates a schematic overview of CAF exosome signaling during gemcitabine treatment. Gemcitabine treatment leads to upregulation of Snai1 and miR-146a as well as exosome secretion in CAFs that could lead to increased cell proliferation, tumor growth, and chemoresistance of adjacent cancer epithelial cells. GW4869, for example, suppresses exosome release and therefore exosomal transfer of Snai1 and miR-146a.

FIG. 13A illustrates a graph of relative copy number of microRNA derived from extracellular vesicles secreted from 1) gemcitabinetreated PCAFs (Exo GEM) and 2) PBS-treated PCAFs (Exo Cont), as quantified via RT-PCR. MicroRNA levels were normalized to number of cultured cells and volume of cell-conditioned media. FIG. 13B illustrates a graph of relative copy number of MicroRNA, again quantified as previously done via RT-PCR utilizing extracellular vesicle RNA secreted from a second patient-derived PCAF cell line (CAF2).

FIG. 14A illustrates a graph of MicroRNA-92a levels that are artificially elevated in pancreatic cancer epithelial cells by transfecting the cells with microRNA-92a mimic nucleotides. Elevated levels of microRNA-92a in transfected cells compared to cells transfected with a negative scramble control siRNA was verified via RT-PCR. FIG. 14B illustrates a graph of the results of cells transfected with miR-92a mimic showed reduced levels of PTEN mRNA, as established by RT-PCR, validating that miR-92a binds to and degrades PTEN mRNA.

FIG. 15A illustrates a Western blot of the results of epithelial cancer cells (AsPC1) grown in the presence of control media, PCAF-conditioned media, or exosome depleted (ED) PCAF-conditioned media. Upon growth in PCAF-conditioned media PTEN protein levels decreased. Depletion of exosomes from PCAFconditioned media restored PTEN protein levels. FIG. 15B illustrates a Western blot of phospho-AKT protein levels increasing upon growth in PCAF-conditioned media. Depletion of exosomes from PCAF-conditioned media restored phospho-AKT protein levels.

FIG. 16 illustrates a graph of the results from NOD/SCID mice being subcutaneously implanted with one million AsPC1 cells and 200,000 CAF1 cells. Two weeks post implantation mice were treated intraperitoneally with DMSO+PBS, DMSO+gemcitabine, PBS+GW4869, or gemcitabine+GW4869 twice weekly for two weeks. Tumors were extracted for RNA and PTEN expression within tumor samples was quantified via RT-PCR.

FIG. 17A illustrates a graph of the results from pancreatic cancer epithelial cells (AsPC1) grown in PCAF-conditioned media; the cells expressed higher levels of microRNA-92a compared to control media, as established via RTPCR. FIG. 17B illustrates a graph of the results from pancreatic cancer epithelial cells (AsPC1) co-cultured with CAFs treated with GW4869; these cells demonstrated reduced expression of microRNA-92a compared to AsPC1 cells co-cultured with CAFs treated with DMSO as a control.

FIG. 18 illustrates a schematic overview. Gemcitabine treatment causes increased secretion of exosomes from PCAFs as well as increased secretion of miR-21, miR-221, miR-222, miR-181a, and miR92a within the exosomes which can all decrease tumor suppressor, PTEN, expression, leading to increased cell proliferation, tumor growth, and chemoresistance of pancreatic cancer epithelial cells which take up those exosomes. GW4869 suppresses exosome release and therefore exosome transfer of these microRNAs.

FIG. 19 illustrates a graph of the results from pancreatic cancer epithelial cells being treated with DMSO as a control or 2011M GW4869 for one day then treated with gemcitabine for three days. Live and dead cells were quantified using an automated cell counter and trypan blue staining. Percent live cells was quantified.

FIG. 20 illustrates a graph of the results from pancreatic cancer epithelial cells being grown to full confluency and placed in serum-free media to prevent cell proliferation. A scratch was made down to center of the plate to create a cell free zone for migration. Cells were then given DMSO as a control or 2011M GW4869. Images were taken each day for three days and distance of cell migration was quantified using ImageJ.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

### Overview of Cancer Treatment Methods

As provided above, pancreatic ductal adenocarcinoma (PDAC) has a dismal 5-year survival rate of less than 7%. PDAC is currently the fourth leading cause of cancer-related deaths in the United States and is predicted to become the second leading cause of cancer-related deaths by 2030. The poor prognosis of the disease is associated with late detection, aggressive tumor biology, and poor response to available therapies. Gemcitabine, a nucleoside analog, is the standard chemotherapeutic agent for adjuvant therapy of resectable PDAC and a commonly used agent in other treatment settings, including neoadjuvant treatment of borderline resectable PDAC and palliative treatment of metastatic PDAC. Despite gemcitabine being one of the most commonly used, single chemotherapeutic agents in pancreatic cancer, response rates are poor. The vast majority of patients (e.g., 74% of patients in an observed cohort) receiving adjuvant gemcitabine eventually show tumor recurrence. This dismal prognosis shows an urgent need for greater understanding of the complete biology of PDAC in order to develop effective therapeutic strategies.

During tumorigenesis stromal tissue, comprising fibroblasts, increases in volume surrounding sites of hyperplasia, metaplasia, dysplasia, inflammation, and neoplasia. Higher than normal number and volume of fibroblast cells surround early precursor cellular lesions known as pancreatic intraepithelial neoplasias (PanINs) and acinar to ductal metaplasias (ADMs), both of which are thought to be the original sites of cancer cell development and tumor formation. While tumorigenesis persists, more fibroblasts surround the cancer tissue, and pancreatic ductal adenocarcinoma (PDAC) tumors end up largely consisting of stromal tissue and fibroblasts. Even though approximately 90% of PDAC tumor volume consists of stromal tissue, current therapies focus predominantly on targeting the proliferation of the rapidly growing epithelial cancer cells.

MicroRNAs are short, single stranded, RNA nucleotide sequences ranging from approximately 18-25 nucleotides long. MicroRNAs that have a complementary base sequence to that of an mRNA sequence at the 3′ untranslated region (UTR) have the ability to bind to the 3′ UTR and inhibit translation of the mRNA into an amino acid sequence. Further, if the microRNA is a strong complementary match, it may assist with the degradation of the mRNA strand upon binding to the mRNA strand. Therefore microRNAs can be regulators of genetic translation that can alter cell behavior, such as proliferation, cell growth, cell senescence, cell migration, etc.

MicroRNAs can be secreted from cells through extracellular vesicles such as microvesicles and exosomes. Therefore, microRNAs can be found in bodily fluids such as blood, lymphatic fluid, saliva, urine, and breast milk where they are mostly found encased in the lipid extracellular vesicles and are protected from degradation by RNases. Cancer cells secrete more exosomes and microvesicles from the cell surface than normal cells. Cancer cells can also secrete more of certain microRNAs within these vesicles compared to normal cells, and they can change their microRNA secretion upon drug treatment.

Overall, gemcitabine, the standard of care chemotherapeutic, does not significantly increase overall survival time of pancreatic cancer patients. Moreover, gemcitabine has already proven to be a paradoxical drug as it not only promotes expression of the Snai1 transcription factor (encoded by SNAI1), but also triggers NF-κB activation, CXCR4 expression, induction of reactive oxygen species, upregulation of cancer-stem cell markers and AKT activity. Together, these undesired side effects lead to increased chemoresistance and cell motility. As reported in Richards et al., 2016, which is incorporated herein by reference in its entirety and appended hereto in Appendix A, gemcitabine treatment causes cancer-associated fibroblasts (CAFs) to greatly increase the secretion of chemoresistance promoting exosomes.

Exosomes, specifically, are secreted lipid vesicles that range in size from 20-150 nm in diameter and contain microRNA, DNA, mRNA, proteins, and other molecules. However, the definition for exosomes is still ambiguous and changes as the years progress. Yet, most extracellular vesicle studies use the term “exosomes” to describe the molecules they study. Therefore, while “exosome” is the common term used in the field and will be the term most used in this application, it will, in the context of this application, refer to extracellular vesicles, defined as lipid vesicles secreted and taken up by cells that contain various cellular molecules such as proteins, microRNA, DNA, and RNA.

### Problem to be Solved

Recent studies have shown that exosomes released from fibroblasts have been found to increase invasive behavior and therapy resistance pathways in breast cancer cells. Other studies have shown that exosomes that travel to distant organs through the circulation system are key to altering the microenvironment in a way that supports tumor growth. However, no previous study examined the impact of exosomes on chemoresistance of pancreatic cancer cells. No previous study determined the impact of exosomes on pancreatic cancer cell proliferation. No previous study examined the impact of gemcitabine or nab-paclitaxel treatment on pancreatic cancer cell exosome release and exosome cargo. No previous study examined the role of pancreatic cancer-associated fibroblast derived exosomes on cancer cell behavior, particularly pancreatic cancer cell behavior. No previous study examined the impact of exosome secretion inhibition on pancreatic cancer cell proliferation or chemoresistance. No previous study specifically examined the impact of inhibition of CAF exosome secretion on pancreatic cancer cell proliferation or chemoresistance. Further, no previous study used an exosome secretion inhibitor in vivo to examine its impact on pancreatic tumor growth.

Pancreatic cancer, as well as other cancers, are very resistant to chemotherapy drug treatment. The vast majority of patients (e.g., 74% of a measured cohort) receiving adjuvant gemcitabine eventually show tumor recurrence. Only 15-20% of patients that are diagnosed during early stages of disease and qualify for surgery to remove to primary tumor along with chemotherapy treatment survive past five years due to recurrence of chemoresistant tumors.

Accordingly, additional methods for treating cancer are desired, particularly methods that address the chemoresistance, tumor growth, and/or metastasis of cancer cells.

### Exemplary Solutions to the Problem

The present disclosure addresses the foregoing and surprisingly demonstrates that exosome secretion inhibitors decrease chemoresistance of cancer cells, suppress proliferation of cancer cells, suppress the migration of cancer cells, and/or reduce tumor growth in vivo.

As demonstrated herein, gemcitabine treatment and nab-paclitaxel treatment, itself, increases exosome secretion from cells, contributes, at least in part, to chemoresistance in vivo. Further, these exosomes increase proliferation, migration, and chemoresistance of pancreatic cancer cells. Exosomes derived from gemcitabine-treated cells increase proliferation and chemoresistance more than exosomes derived from untreated cells. Moreover, gemcitabine treatment is shown herein to result in the increase of secretion of certain microRNAs and Snai1 mRNA within exosomes, contributing to the chemoresistance of cancer cells.

Embodiments of the present disclosure enable cancer treatment methods that utilize a chemical and/or molecular inhibitor of extracellular vesicle signaling, which without being bound to a specific theory of action, can inhibit exosome secretion and/or exosome uptake. Exemplary treatment methods include administering an exosome inhibitor and/or a patient receiving an exosome inhibitor. Such exemplary treatment methods could be utilized in patients, alone, but in some instances an exosome inhibitor is used in combination with chemotherapy. The cancer treatment methods disclosed herein beneficially act to suppress one or more of chemoresistance, tumor growth, or metastasis. In some instances, the cancer treatment method is for treating pancreatic cancer.

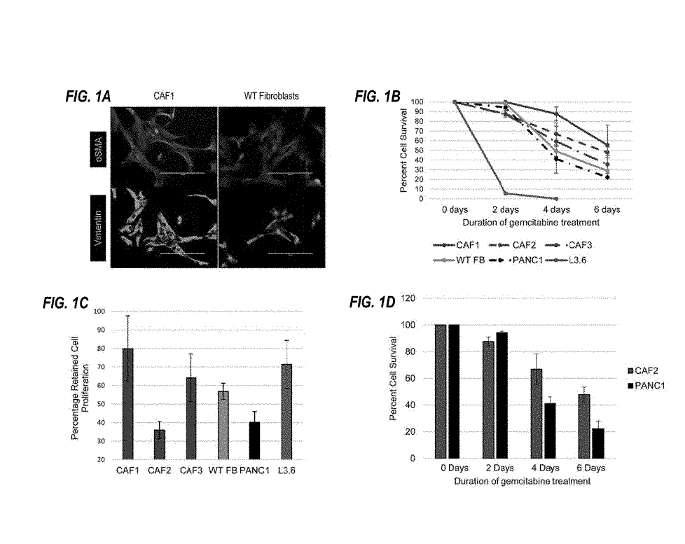

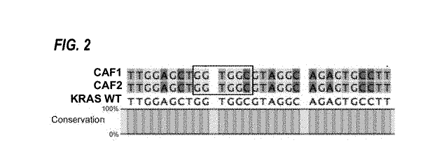

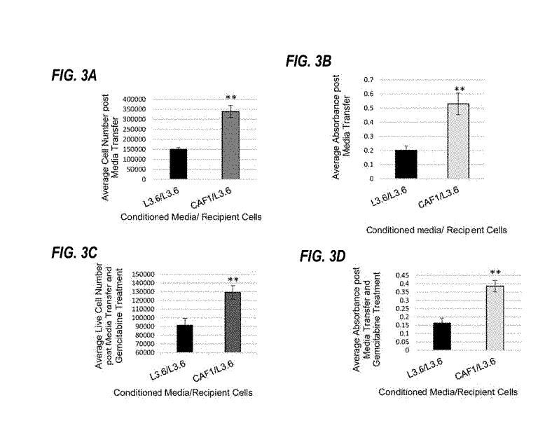

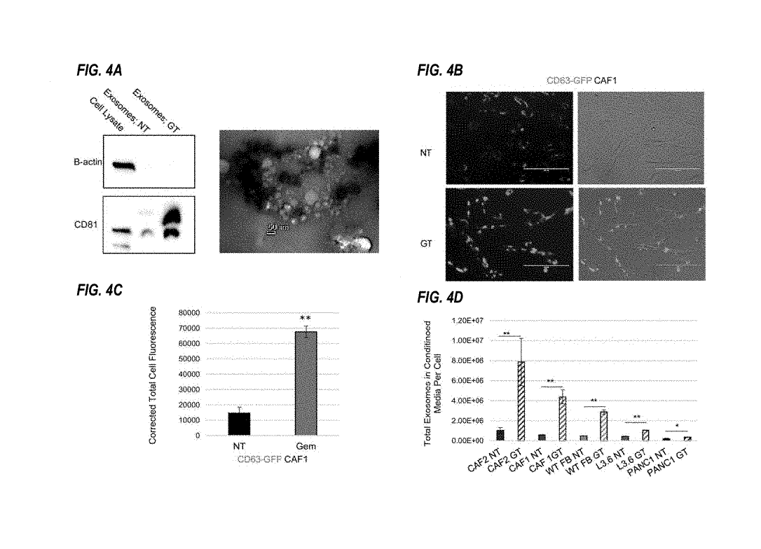

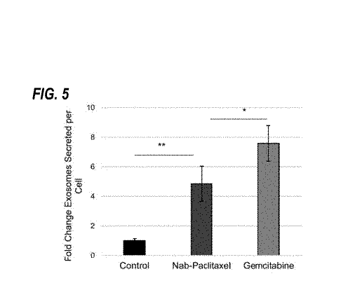

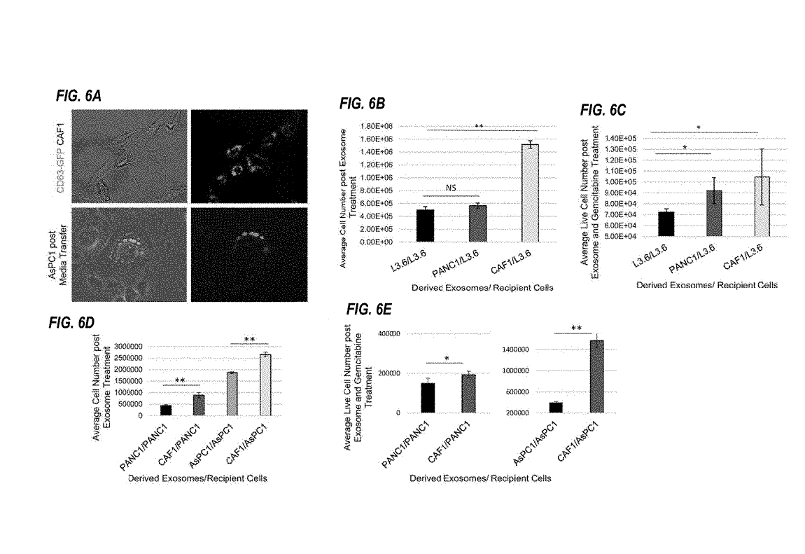

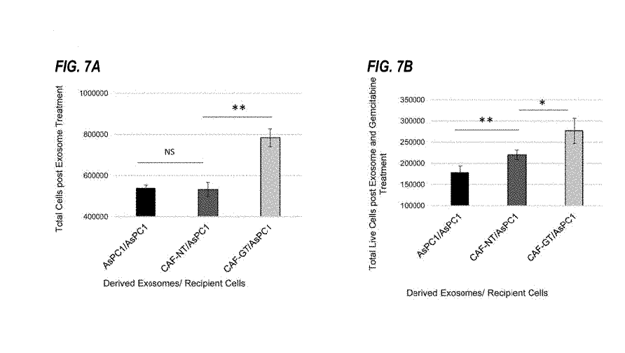

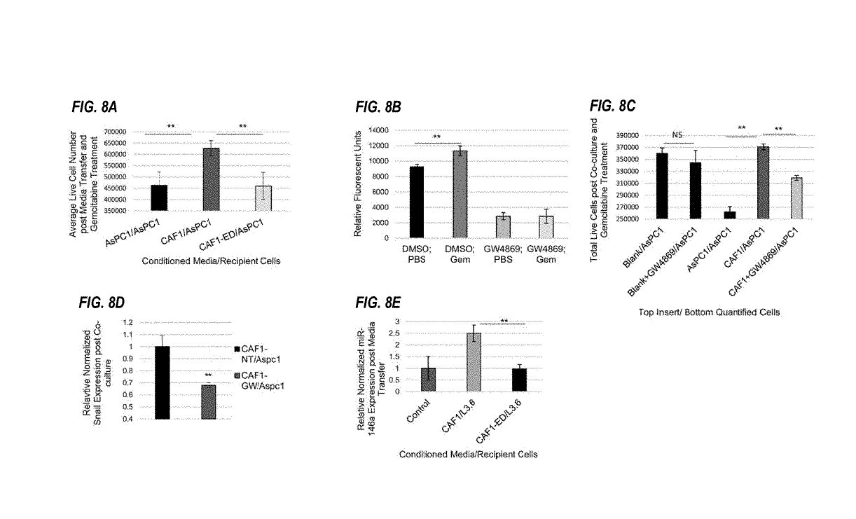

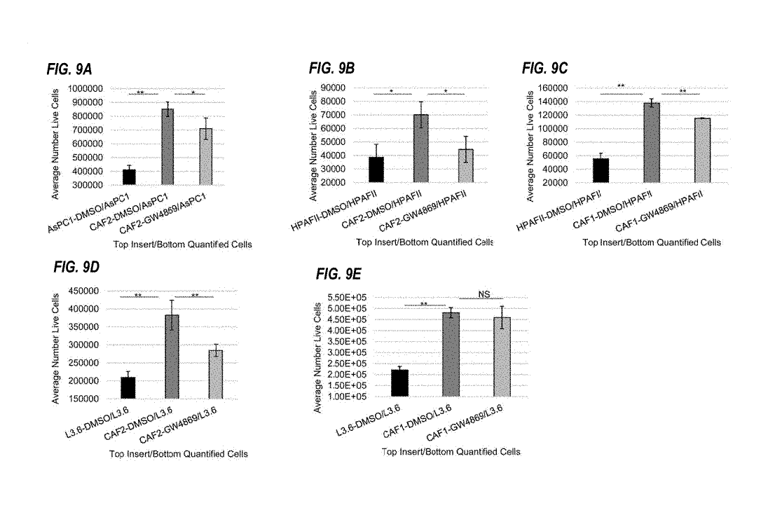

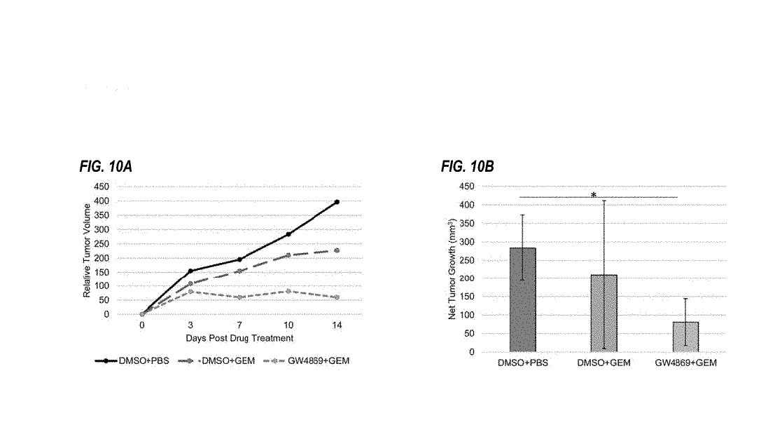

Patient-derived pancreatic cancer-associated fibroblasts (PCAFs) are innately chemoresistant (FIG. 1). PCAFs were stained for fibroblast markers and sequenced to determine that they are indeed fibroblasts (FIG. 1; FIG. 2). PCAF conditioned cell media increased proliferation and chemoresistance of a chemosensitive PDAC cell line (FIG. 3), gemcitabine chemotherapeutic as well as nab-paclitaxel increases secretion of exosomes from cells (FIG. 4; FIG. 5), most prominently from PCAFs, and PCAF exosomes increased proliferation and chemoresistance of pancreatic epithelial cancer cells (PECCs) (FIG. 6). Further, exosomes derived from gemcitabine-treated PCAFs increased proliferation and chemoresistance of PECCs more than exosomes derived from untreated PCAFs (FIG. 7). Inhibiting secretion of exosomes from PCAFs with the exosome secretion inhibitor, GW4869, while PCAFs were co-cultured with PECCs, reduced the chemoresistance of the PECCs (FIG. 8; FIG. 9). Further, treating mice with GW4869 reduced tumor growth (FIG. 10).

Gemcitabine was previously shown to upregulate expression of the transcription factor Snai1 (SNAI1) in PDAC cancer cells. This upregulation may be mediated in part by NF-κB that is activated by gemcitabine and stabilizes Snai1 as well as promotes Snai1 expression. Snai1, is a marker of epithelial-to-mesenchymal transition (EMT), promotes tumor progression and chemoresistance, and is correlated with poor prognosis. Snai1 was previously shown to activate transcription of microRNA-146a. MiR-146a is highly upregulated in pre-malignant disease as well as metastatic PDAC compared to normal samples and was also found upregulated in chemoresistant PDAC cancer stem cells.

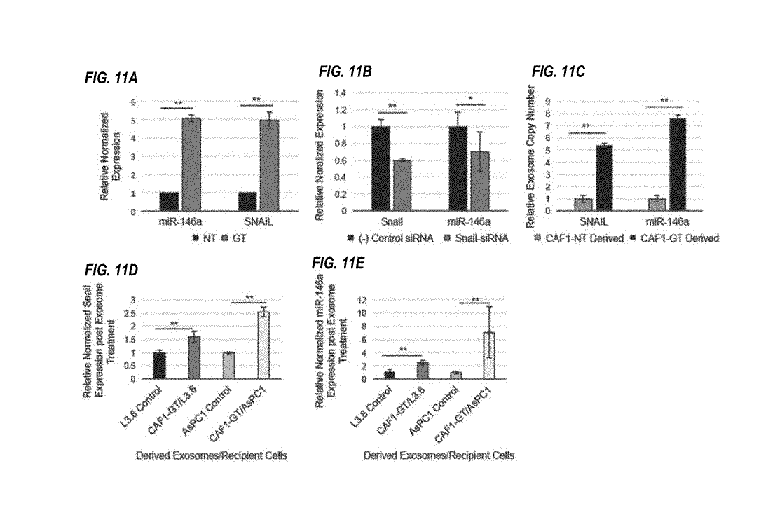

As provided herein, levels of both miR-146a and Snai1 mRNA are higher in exosomes secreted from gemcitabine-treated PCAFs than untreated PCAFs (FIG. 11). Further, these gemcitabine-treated PCAF exosomes increased the levels of both Snai1 mRNA and miR146a in cells receiving the exosomes (FIG. 11). When exosome secretion from PCAFs was inhibited by GW4869, the cellular level of Snai1 mRNA within co-cultured PECCs decreased (FIG. 8). When exosomes were depleted from PCAF-conditioned media, PECCs cultured in the conditioned-media displayed reduced chemoresistance and reduced cellular miR-146a levels (FIG. 8).

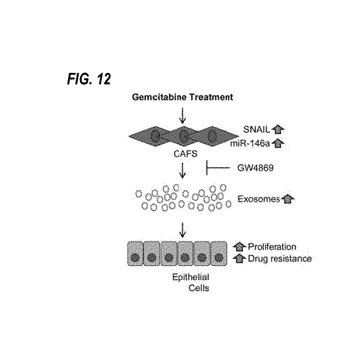

Overall, these data indicate that 1) PCAF secreted exosomes enhance chemoresistance and proliferation of PECCs 2) gemcitabine treatment enhances both exosome secretion from PCAFs as well as secretion of factors within exosomes that can promote chemoresistance 3) exosome-induced chemoresistance may be mediated through factors such as, but not limited, to Snai1 and miR-146a, and 4) exosome inhibitors may help alleviate exosome signaling induced chemoresistance (FIG. 12).

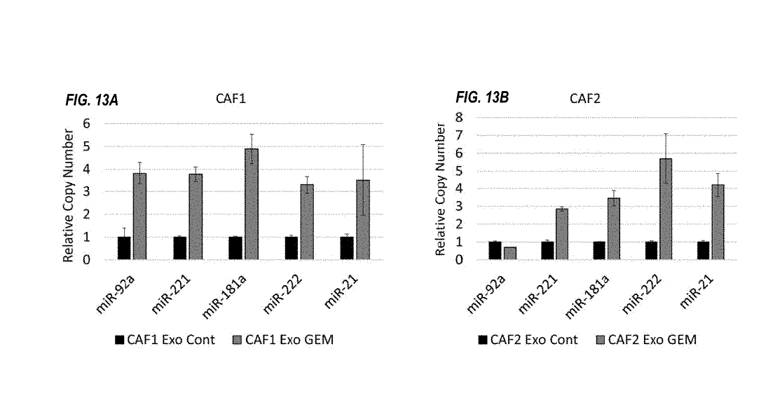

While Snai1 and miR-146a may play an important role in promoting chemoresistance of PECCs, other potential mechanisms exist in which PCAF exosomes may increase chemoresistance. For example, by treating PCAFs with PBS as a control or gemcitabine, collecting their secreted extracellular vesicles, lysing them, collecting the microRNA within, and quantifying the microRNAs, the role of microRNAs is made clearer. MicroRNA-Seq results show that when normalizing to total RNA microRNAs hsa-miR-21, hsa-miR-221, hsa-miR-222, hsa-miR-181a, and hsa-miR-92a were more prevalent in exosomes derived from gemcitabine-treated PCAF derived exosomes compared untreated PCAF derived exosomes (TABLE 1). These results were validated via RT-PCR (FIG. 13).

TABLE 1
PCAFs were treated with PBS as a control or gemcitabine. PCAF
conditioned-media was collected thereafter, and extracellular
vesicles were isolated from the conditioned-media. RNA was isolated
from the extracellular vesicles. MicroRNA-Seq was performed utilizing
the RNA isolated from the extracellular vesicles, and results
were normalized to total RNA. Analysis from the microRNA-Seq
shows increased prevalence of five microRNAs within extracellular
vesicle populations derived from gemcitabine-treated PCAFs (GEM)
compared to PBS-treated PCAFs (NT).

| | | Fold Change | |
|---|---|---|---|
| microRNA | SEQ ID | (GEM vs. NT) | p-value |
| hsa-miR-21-5p | SEQ ID NO: 6 | 2.530826725 | 0.003991 |
| hsa-miR-181a-5p | SEQ ID NO: 7 | 3.508503757 | 0.0132411 |
| hsa-m1R-221-3p | SEQ ID NO: 8 | 4.324901312 | 0.0064948 |
| hsa-miR-222-3p | SEQ ID NO: 9 | 2.648685853 | 0.0426011 |
| hsa-miR-92a-3p | SEQ ID NO: 10 | 4.65992105 | 0.0042333 |

These microRNAs can alter cellular pathways involved in tumorigenesis and can target tumor suppressor gene, PTEN (TABLE 2; TABLE 3).

TABLE 2
Putative gene targets and manipulated pathways of hsa-miR-21, hsa-miR-221, hsamiR-222,
hsa-miR-181a, and hsa-miR-92a were determined via DIANA TOOLS mirPath software.

| Pathways Manipulated | |
|---|---|
| by all 5 microRNAs | |
| (miR- 21-5p, miR-92a-3p, | |
| miR-221-3p, miR-181a-5p, | |
| and miR-222-3p) | Putative Gene Targets |
| Wnt Signaling Pathway | GSK3B, PPP2R5E, LRP6, TCF4, WNT5A, FZD6, SKP1, FRAT2, |
| | CAMK2A, NLK, SENP2, FZD10, AXIN2, CXXC4, WIF1, NFATC3, |
| | DAAM1, TBL1XR1 |
| MAPK Signaling Pathway | FOS, NTF3, RASA2, CRK, CACNB4, RAP1A, FASLG, TAOK1, |
| | CACNA1I, NLK, DUSP10, RASGRP1, PPM1A, DUSP8, CDC42, |
| | FGF18, RPS6KA3, AKT3, MAP2K1, STMN1, MAP3K2, MEF2C, |
| | DUSP5, MAP2K4, RAP1B, TGFBR2 |
| PI3K-AKT Signaling | PHLPP2, PRLR, GSK3B, TSC1, PPP2R5E, ITGA8, PIK3CB, |
| | CREB5, YWHAG, COL27A1, ANGPT2, ITGA5, PIK3AP1, CDKN1B, |
| | FASLG, ITGAV, DDIT4, PIK3R3, COL5A1, KIT, PIK3R1, JAK3, |
| | FGF18, COL1A2, AKT3, PIK3CA, MAP2K1, ITGA6, PTEN, SGK3, |
| | KDR, SPP1, BCL2L11, RPS6KB1, COL4A1, IL6R |
| HIF-1 Signaling | STAT3, PIK3CB, ARNT, ANGPT2, CDKN1B, CAMK2A, PIK3R3, |
| | PIK3R1, AKT3, PIK3CA, MAP2K1, PDHB, PFKFB4, RPS6KB1, |
| | EGLN1, IL6R |
| Focal Adhesion | GSK3B, CRK, ITGA8, PIK3CB, PIP5K1C, RAP1A, COL27A1, ITGA5, |
| | VCL, ITGAV, PPP1R12A, PIK3R3, COL5A1, PIK3R1 |
| | CDC42, COL1A2, AKT3, PIK3CA, MAP2K1, ITGA6, PTEN, KDR, |
| | RAP1B, SPP1, COL4A1 |
| T cell Receptor Signaling | FOS, GSK3B, PIK3CB, PIK3R3, RASGRP1, PIK3R1, CDC42, AKT3, |
| | CD4, PIK3CA, MALT1, MAP2K1, CARD11, NFATC3 |
| VEGF Signaling | PIK3CB, PIK3R3, PTGS2, PIK3R1, CDC42, AKT3, PIK3CA, |
| | MAP2K1, NFATC3, KDR |
| Endocytosis | CHMP7, DNAJC6, GRK5, GRK7, PIP5K1C, PDCD6IP, EEA1, |
| | ITCH, VPS36, ASAP1, ZFYVE16, RAB11FIP2, GIT2, SMURF1, |
| | RAB11A, KIT, CDC42, SMAD7, RAB11FIP1, KDR, TGFBR2, |
| | ADRB1 |
| Jak-Stat Pathway | PRLR, STAT3, PIK3CB, CSF2RB, CNTFR, SPRED2, PIK3R3, LIFR, |
| | PIK3R1, JAK3, SPRY1, PIAS4, AKT3, PIK3CA, SPRY2, IL6R |
| Endoplasmic Reticulum | UBE2E3, SAR1B, YOD1, HSPA5, UBE2J1, SKP1, EDEM1, |
| Protein Processing | EDEM3, MAN1A2, SVIP, SEC62, SEC24A, LMAN1, UBE2D3, |
| | SEC24B, UBE2G1, DNAJB12, DERL1, ATXN3, RRBP1, PARK2 |
| Ubiquitin Mediated | UBE2E3, WWP2, FBXW7, ITCH, UBE2J1, SKP1, CUL5, SKP2, |
| Proteolysis | BIRC6, SMURF1, PIAS4, UBE2D3, CDC27, UBE2G1, UBE2W, |
| | PARK2 |
| B Cell Receptor Signaling | FOS, GSK3B, PIK3CB, PIK3AP1, PIK3R3, PIK3R1, AKT3, |
| | PIK3CA, MALT1, MAP2K1, CARD11, NFATC3 |

TABLE 3
Predicted targeted 3′ UTR position of PTEN mRNA transcript and microRNA
target gene score (miTG score) was determined for each of hsa-miR-21, hsa-miR-
221, hsa-miR-222, hsa-miR-181a, and hsa-miR-92a utilizing microT-CDS software.

| | | Predicted Targeted 3′UTR Position of | miTG |
|---|---|---|---|
| microRNA | SEQ ID | PTEN Transcript | Score |
| hsa-miR-21-5p | SEQ ID NO: 6 | 1588-1607; 4789-4814 | 0.406 |
| hsa-miR-181a-5p | SEQ ID NO: 7 | 1261-1287; 1869-1887; 2255-2282; 2289-2316; | 0.882 |
| | | 2780-2801; 3923-3933; 4680-4700; 5114-5137; | |
| | | 5249-5275; 5881-5908; 6194-6220 | |
| hsa-m iR-221-3p | SEQ ID NO: 8 | 180-205; 2668-2685; 4381-4408; 5908-5928 | 0.755 |
| hsa-miR-222-3p | SEQ ID NO: 9 | 180-205; 2668-2685; 4381-4408; 5908-5928 | 0.812 |
| hsa-miR-92a-3p | SEQ ID NO: 10 | 59-84; 2842-2865; 3987-4009 | 0.971 |

Previous studies identify all five of the microRNAs to increase cell proliferation, migration, metastasis, and tumor growth (TABLE 4).

TABLE 4
Literature search was performed to determine the effect of increased levels
of hsa-miR-21, hsa-miR-221, hsa-miR-222, hsa-miR-181a, or hsa-miR-92a on
cell behavior, tumor behavior, human prognosis, and PTEN expression.

| Effect on Cancer Biology | MicroRNAs | CORRESPONDING SEQ ID |
|---|---|---|
| Increased Proliferation | miR-21, miR-92a, | SEQ ID NO: 3, SEQ ID NO: 10, SEQ ID |
| | miR-221/222, miR-181a | NO: 8/SEQ ID NO: 9, SEQ ID NO: 7 |
| Increased Migration | miR-21, miR-92a, | SEQ ID NO: 3, SEQ ID NO: 10, SEQ ID |
| | miR-221/222, miR-181a | NO: 8/SEQ ID NO: 9, SEQ ID NO: 7 |
| Inhibited Apoptosis | miR-21, miR-92a, | SEQ ID NO: 3, SEQ ID NO: 10, SEQ ID |
| | miR-221/222, miR-181a | NO: 8/SEQ ID NO: 9, SEQ ID NO: 7 |
| Increased Tumor Growth | miR-21, miR-92a, | SEQ ID NO: 3, SEQ ID NO: 10, SEQ ID |
| | miR-221/222, miR-181a | NO: 8/SEQ ID NO: 9, SEQ ID NO: 7 |
| Increased Metastasis | miR-21, miR-221/222, | SEQ ID NO: 3, SEQ ID NO: 8/SEQ ID |
| | miR-181a | NO: 9, SEQ ID NO: 7 |
| Poor Prognosis | miR-21, miR-92a, | SEQ ID NO: 3, SEQ ID NO: 10, SEQ ID |
| | miR-221/222, miR-181a | NO: 8/SEQ ID NO: 9, SEQ ID NO: 7 |
| Upregulated in Primary or | miR-21, miR-92a, | SEQ ID NO: 3, SEQ ID NO: 10, SEQ ID |
| Metastatic Tumors | miR-221/222, miR-181a | NO: 8/SEQ ID NO: 9, SEQ ID NO: 7 |
| Suppresses PTEN | miR-21, miR-92a, | SEQ ID NO: 3, SEQ ID NO: 10, SEQ ID |
| | miR-221/222, miR-181a | NO: 8/SEQ ID NO: 9, SEQ ID NO: 7 |

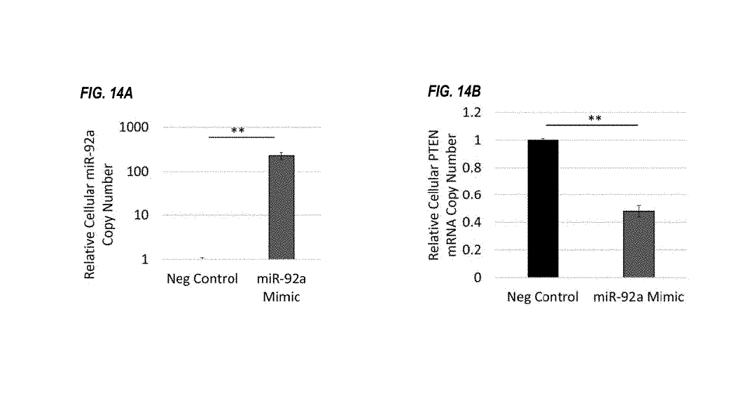

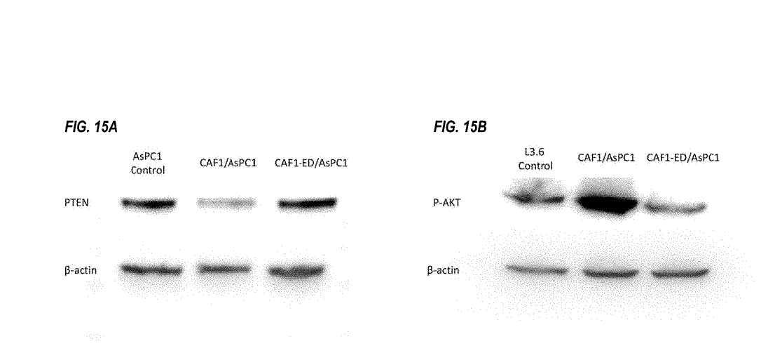

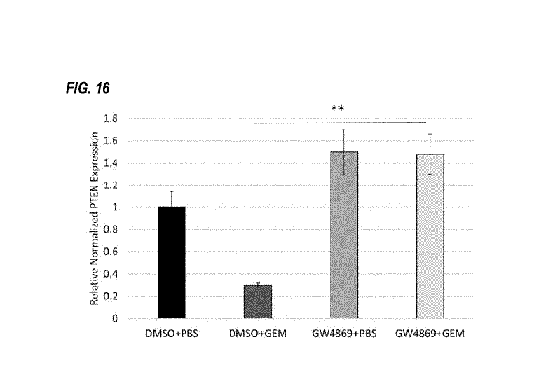

Certain microRNAs were transfected into pancreatic epithelial cancer cells and were validated to target tumor suppressor gene, PTEN via RT-PCR (FIG. 14). When pancreatic cancer epithelial cells were cultured in PCAF-conditioned media, PTEN protein levels were decreased compared to control, and PTEN protein levels were restored when PCAF-conditioned media was depleted of exosomes (FIG. 15). PTEN acts as a tumor suppressor in part by inhibiting phosphorylation of AKT. When AKT is phosphorylated it becomes activated and promotes cell proliferation, protein synthesis, cell growth, cell metastasis, and chemoresistance. When pancreatic cancer epithelial cells were cultured in PCAF-conditioned media, phosphorylated AKT protein levels were increased compared to control, and phosphorylated-AKT protein levels were reduced when PCAF-conditioned media was depleted of exosomes (FIG. 15). Further, when tumor-bearing mice were treated with gemcitabine, PTEN expression within the tumors decreased compared to control. However, when the mice were treated with both gemcitabine and exosome inhibitor, GW4869, PTEN expression levels within the mice tumors was increased and restored (FIG. 16).

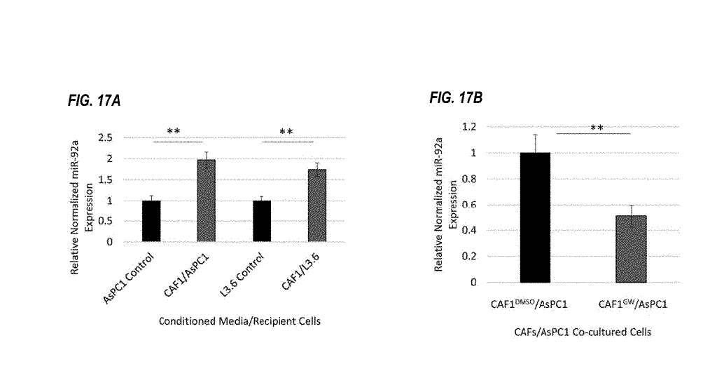

Additionally, when pancreatic epithelial cancer cells were grown in PCAF-conditioned media, expression level of miR-92a was increased. However, when the PCAFs were treated with exosome inhibitor, GW4869, expression of the microRNA within co-cultured PECCs decreased (FIG. 17).

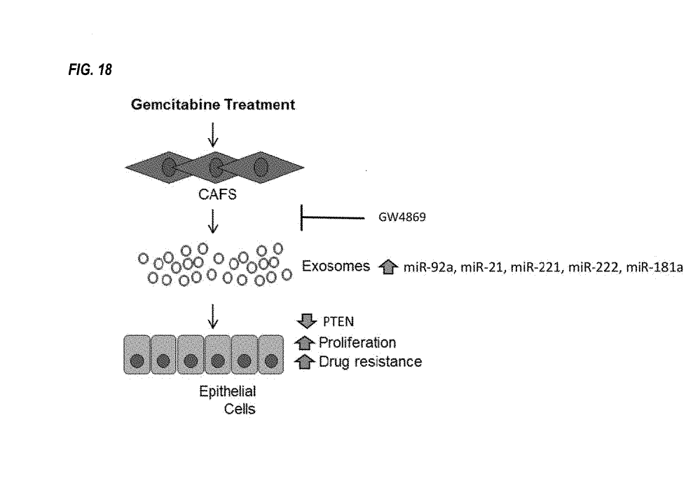

These data suggest that microRNAs hsa-miR-21, hsa-miR-221, hsa-miR-222, hsa-miR181a, and hsa-miR-92a are more prevalent within PCAF derived exosomes after gemcitabine treatment, can be transferred to pancreatic cancer cells, can inhibit translation of PTEN, and can promote chemoresistance and proliferation. Depletion of exosomes from PCAF conditioned-media increases and restores PTEN expression within PECCs and reduces phospho-AKT levels within PECCs. Inhibition of exosome secretion from PCAFs reduces miR-92a expression in PECCs and increases PTEN expression within tumors. These data represent a novel mechanism in which PCAF exosomes and gemcitabine treatment may promote chemoresistance and proliferation in PECCs, and a novel treatment therapy to reduce the increase in chemoresistance, using an exosome secretion inhibitor (FIG. 18).

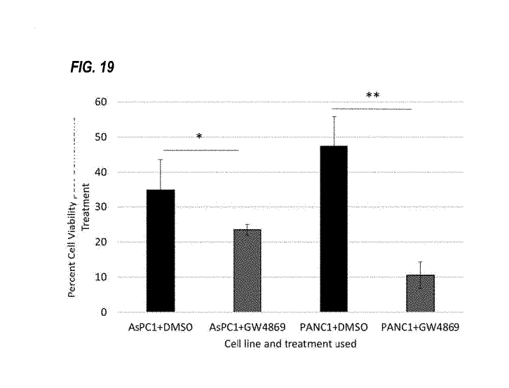

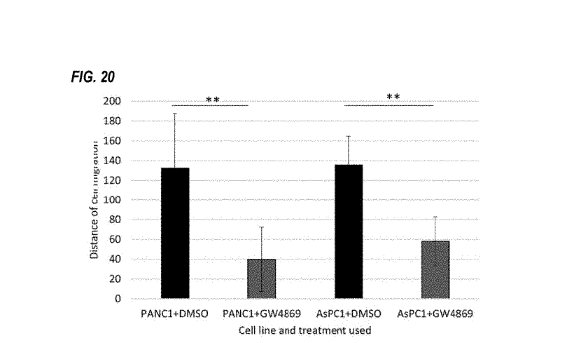

In some embodiments, treatment of PECCs with GW4869 demonstrates reduced chemoresistance and reduced cell migration compared to control PECCs treated with DMSO (FIG. 19; FIG. 20). These data suggest that alteration of PECC cell behavior is not dependent on PCAF exosomes, but inhibiting exosome secretion from the PECCs, themselves, also reduces chemoresistance as well as cell migration of the PECCs.

In an exemplary embodiment, the use of GW4869 reduces extracellular vesicle secretion from cancer cells and/or cancer-associated cells, particularly pancreatic cancer epithelial cells and pancreatic cancer-associated fibroblasts. In some embodiments, GW4869 is dissolved in DMSO prior to administration in a subject. However, in some embodiments, GW4869 or other drugs can be dissolved in a more human-friendly, less toxic dissolvent such as PBS or sodium citrate. It should be appreciated that GW4869, other inhibitors of extracellular vesicle signaling, and/or chemotherapeutic drugs can be prepared as a pharmaceutical composition, as described herein.

Additionally, it should be appreciated that dosages can be determined as described herein. In one embodiment, the dosage of pharmaceutical composition depends on the drug itself, its efficacy, and its toxicity. As an exemplary embodiment, 2-2.5 gg GW4869 per gram mouse body weight can be intraperitoneally injected into mice twice weekly for two weeks to achieve therapeutic results.

### Abbreviated List of Defined Terms

To assist in understanding the scope and content of the foregoing and forthcoming written description and appended claims, a select few terms are defined directly below.

As used herein, the term “cancer” includes solid tumor carcinomas such as, but not limited to, colorectal cancer, pancreatic cancer, breast cancer, lung cancer, prostate cancer, etc.

The term “extracellular vesicle,” as used herein, refers to lipid molecules which contain protein, RNA, DNA or any combination thereof and are actively secreted and taken up by cells. The term “extracellular vesicles” is intended to include literature terms such as “exosomes,” “microvesicles,” “oncosomes,” and “ectosomes.”

Inhibitors of extracellular vesicle signaling refers to chemical or molecular compounds which suppress the uptake of extracellular vesicles by cells, or which suppress secretion or release of extracellular vesicles from cells. Such examples of extracellular vesicle secretion inhibitors include GW4869, Cytochalasin D, manumycin-A, spiroepoxide, dimethyl amiloride, siRNAs/shRNAs targeting neutral sphingomyelinase-2 or Rab27a. Examples of potential inhibitors of extracellular vesicle uptake can be found in Mulcahy et al., 2014, Journal of Extracellular Vesicles, incorporated herein by reference in its entirety.

### Pharmaceutical Compositions

While it is possible for the compounds described herein to be administered alone, it may be preferable to formulate the compounds as pharmaceutical compositions (e.g., formulations). As such, in yet another aspect, pharmaceutical compositions useful in the methods and uses of the disclosed embodiments are provided. A pharmaceutical composition is any composition that may be administered in vitro or in vivo or both to a subject to treat or ameliorate a condition. In a preferred embodiment, a pharmaceutical composition may be administered in vivo. A subject may include one or more cells or tissues, or organisms. In some exemplary embodiments, the subject is an animal. In some embodiments, the animal is a mammal. The mammal may be a human or primate in some embodiments. A mammal includes any mammal, such as by way of non-limiting example, cattle, pigs, sheep, goats, horses, camels, buffalo, cats, dogs, rats, mice, and humans.

As used herein the terms “pharmaceutically acceptable” and “physiologically acceptable” mean a biologically compatible formulation, gaseous, liquid or solid, or mixture thereof, which is suitable for one or more routes of administration, in vivo delivery, or contact. A formulation is compatible in that it does not destroy activity of an active ingredient therein (e.g., an inhibitor of extracellular vesicle signaling and/or other chemotherapeutic drug) or induce adverse side effects that outweigh any prophylactic or therapeutic effect or benefit.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH and may range from a pH of about 3 to a pH of about 11, preferably about pH 3 to about pH 7, depending on the formulation and route of administration. In alternative embodiments, it may be preferred that the pH is adjusted to a range from about pH 5 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein or may include a second active ingredient useful in the treatment or prevention of cancer (e.g., a chemotherapeutic drug).

Formulations, for example, for parenteral or oral administration, are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, and the like.

Compositions may contain one or more excipients. Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences, incorporated herein by reference).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions described herein may be formulated in any form suitable for the intended method of administration. When intended for oral use, for example, tablets, troches, lozenges, aqueous or oil suspensions, non-aqueous solutions, dispersible powders or granules (including micronized particles or nanoparticles), emulsions, hard or soft capsules, syrups, or elixirs may be prepared. Compositions intended for oral use may be prepared according to any method known to the art for the manufacture of pharmaceutical compositions, and such compositions may contain one or more agents including sweetening agents, flavoring agents, coloring agents, and preserving agents to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin, or acacia; and lubricating agents, such as magnesium stearate, stearic acid, or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing, or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g., dextran sulfate); glycoaminoglycans and glycosaminoglycan-like compounds (e.g., hyaluronic acid); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters, or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol, or sucrose. Such formulations may also contain a demulcent, a preservative, a flavoring, or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and an isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may be used in the preparation of injectables.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, and complexation.

Many therapeutics have undesirably short half-lives and/or undesirable toxicity. Thus, the concept of improving half-life or toxicity is applicable to various treatments and fields. Pharmaceutical compositions can be prepared, however, by complexing the therapeutic with a biochemical moiety to improve such undesirable properties. Proteins are a biochemical moiety that may be complexed with one or more active ingredients for administration in a wide variety of applications. In some embodiments, one or more active ingredients are complexed with a protein to increase the half-life of the active ingredient(s). Additionally, or alternatively, one or more active ingredients are complexed with a protein to decrease the toxicity of the active ingredient. Albumin is a particularly preferred protein for complexation. In some embodiments, the albumin is fat-free albumin.

With respect to the active ingredients disclosed herein, (e.g., an inhibitor of extracellular vesicle signaling and/or other chemotherapeutic drug), a biochemical moiety for complexation can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In some embodiments, the weight ratio of albumin to active ingredient is about 18:1 or less, such as about 9:1 or less. In some embodiments, the active ingredient is coated with albumin.

Alternatively, or in addition, non-biochemical compounds can be added to the pharmaceutical compositions to reduce the toxicity of the therapeutic and/or improve the half-life. Suitable amounts and ratios of an additive that can reduce toxicity can be determined via a cellular assay. With respect to the active ingredient (e.g., an inhibitor of extracellular vesicle signaling and/or other chemotherapeutic drug), toxicity reducing compounds can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In some embodiments, the toxicity reducing compound is a cocoamphodiacetate such as Miranol® (disodium cocoamphodiacetate). In other embodiments, the toxicity reducing compound is an amphoteric surfactant. In some embodiments, the toxicity reducing compound is a surfactant. In other embodiments, the molar ratio of cocoamphodiacetate to active ingredient is between about 8:1 and 1:1, preferably about 4:1. In some embodiments, the toxicity reducing compound is allantoin.

In some embodiments, a pharmaceutical composition is prepared utilizing one or more sufactants. In an exemplary embodiment, the active ingredient (e.g., an inhibitor of extracellular vesicle signaling and/or other chemotherapeutic drug) is complexed with one or more poloxamer surfactants. Poloxamer surfactants are nonionic triblock copolymers composed of a central hydrophobic chain of polyoxypropylene (poly(propylene oxide)) flanked by two hydrophilic chains of polyoxyethylene (poly(ethylene oxide)). In some embodiments, the poloxamer is a liquid, paste, or flake (solid). Examples of suitable poloxamers include those by the trade names Synperonics, Pluronics, or Kolliphor.

In some embodiments, one or more of the poloxamer surfactant in the composition is a flake poloxamer. In some embodiments, the one or more poloxamer surfactant in the composition has a molecular weight of about 3600 g/mol for the central hydrophobic chain of polyoxypropylene and has about 70% polyoxyethylene content. In some embodiments, the ratio of the one or more poloxamer to active ingredient (e.g., an inhibitor of extracellular vesicle signaling and/or other chemotherapeutic drug) is between about 50 to 1; about 40 to 1; about 30 to 1; about 20 to 1; about 10 to 1; about 5 to 1; about 1 to 1; about 1 to 10; about 1 to 20; about 1 to 30; about 1 to 40; or about 1 to 50. In other embodiments, the ratio of the one or more poloxamer to active ingredient (e.g., an inhibitor of extracellular vesicle signaling and/or other chemotherapeutic drug) is between 50 to 1; 40 to 1; 30 to 1; 20 to 1; 10 to 1; 5 to 1; 1 to 1; 1 to 10; 1 to 20; 1 to 30; 1 to 40; or 1 to 50. In some embodiments, the ratio of the one or more poloxamer to active ingredient (e.g., an inhibitor of extracellular vesicle signaling and/or other chemotherapeutic drug) is between about 50 to 1 to about 1 to 50. In other embodiments, the ratio of the one or more poloxamer to active ingredient (e.g., an inhibitor of extracellular vesicle signaling and/or other chemotherapeutic drug) is between about 30 to 1 to about 3 to 1. In some embodiments, the poloxamer is Pluronic F127.

The amount of poloxamer may be based upon a weight percentage of the composition. In some embodiments, the amount of poloxamer is about 10%, 15%, 20%, 25%, 30%, 35%, 40%, about any of the aforementioned numbers, or a range bounded by any two of the aforementioned numbers or the formulation. In some embodiments, the one or more poloxamer is between about 10% to about 40% by weight of a formulation administered to the patient. In some embodiments, the one or more poloxamer is between about 20% to about 30% by weight of the formulation. In some embodiments, the formulation contains less than about 50%, 40%, 30%, 20%, 10%, 5%, or 1% of active ingredient, or about any of the aforementioned numbers. In some embodiments, the formulation contains less than about 20% by weight of active ingredient (e.g., an inhibitor of extracellular vesicle signaling and/or other chemotherapeutic drug).

The above described poloxamer formulations are particularly suited for the methods of treatment, device coatings, preparation of unit dosage forms (e.g., solutions, mouthwashes, injectables), etc.

In one embodiment, the compounds described herein may be formulated for oral administration in a lipid-based formulation suitable for low solubility compounds. Lipid-based formulations can generally enhance the oral bioavailability of such compounds.

As such, in some embodiments, a therapeutically or prophylactically effective amount of a compound described herein can be combined together with at least one pharmaceutically acceptable excipient selected from the group consisting of medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In an alternative embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

A pharmaceutical composition and/or formulation contains a total amount of the active ingredient(s) sufficient to achieve an intended therapeutic effect.

### Dosages

The pharmaceutical compositions may, for convenience, be prepared or provided as a unit dosage form. Preparation techniques include bringing into association the active ingredient (e.g., an inhibitor of extracellular vesicle signaling and/or other chemotherapeutic drug) and pharmaceutical carrier(s) and/or excipient(s). In general, pharmaceutical compositions are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient (e.g., an inhibitor of extracellular vesicle signaling and/or other chemotherapeutic drug) in a free-flowing form such as a powder or granules, optionally mixed with a binder, lubricant, inert diluent, preservative, surface-active or dispersing agent. Molded tablets may be produced by molding, in a suitable apparatus, a mixture of powdered compound (e.g., an inhibitor of extracellular vesicle signaling and/or other chemotherapeutic drug) moistened with an inert liquid diluent. The tablets may optionally be coated or scored and may be formulated so as to provide a slow or controlled release of the active ingredient therein.

Compounds (e.g., an inhibitor of extracellular vesicle signaling and/or other chemotherapeutic drug), including pharmaceutical compositions can be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (e.g., excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound. Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact with the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

Compounds (e.g., an inhibitor of extracellular vesicle signaling and/or other chemotherapeutic drug) can be administered in accordance with the methods at any frequency as a single bolus or multiple dose e.g., one, two, three, four, five, or more times hourly, daily, weekly, monthly, or annually or between about 1 to 10 days, weeks, months, or for as long as appropriate. Exemplary frequencies are typically from 1-7 times, 1-5 times, 1-3 times, 2-times or once, daily, weekly or monthly. For example, twice weekly for two weeks. Timing of contact, administration ex vivo or in vivo can be dictated by the infection, pathogenesis, symptom, pathology, or adverse side effect to be treated. For example, an amount can be administered to the subject substantially contemporaneously with, or within about 1-60 minutes or hours of the onset of a symptom or adverse side effect, pathogenesis, or vaccination. Long-acting pharmaceutical compositions may be administered twice a day, once a day, once every two days, two times a week, three times a week, twice a week, every 3 to 4 days, or every week depending on half-life and clearance rate of the particular formulation. For example, in an embodiment, a pharmaceutical composition contains an amount of a compound as described herein that is selected for administration to a patient on a schedule selected from: twice a day, once a day, once every two days, three times a week, twice a week, and once a week.

Localized delivery is also contemplated, including but not limited to delivery techniques in which the compound is implanted, injected, infused, or otherwise locally delivered. Localized delivery is characterized by higher concentrations of drug at the site of desired action (e.g., the tumor or organ to be treated) versus systemic concentrations of the drug. Well-known localized delivery forms can be used, including long-acting injections; infusion directly into the site of action; depot delivery forms; controlled or sustained delivery compositions; transdermal patches; infusion pumps; and the like. The active ingredient (e.g., an inhibitor of extracellular vesicle signaling and/or other chemotherapeutic drug) can further be incorporated into a biodegradable or bioerodible material or be put into or on a medical device.

Doses may vary depending upon whether the treatment is therapeutic or prophylactic, the onset, progression, severity, frequency, duration, probability of or susceptibility of the symptom, the type pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the symptom(s) or pathology, and any adverse side effects of the treatment or therapy. The skilled artisan will appreciate the factors that may influence the dosage, frequency, and timing required to provide an amount sufficient or effective for providing a prophylactic or therapeutic effect or benefit. The exact dosage will be determined by the practitioner, in light of factors related to the subject that requires treatment. Dosage and administration are adjusted to provide sufficient levels of the active agent(s) or to maintain the desired effect.

The dosage may range broadly, depending upon the desired effects and the therapeutic indication. Alternatively, dosages may be based and calculated upon the per unit weight of the patient, as understood by those of skill in the art. Although the exact dosage will be determined on a drug-by-drug basis, in most cases, some generalizations regarding the dosage can be made. The systemic daily dosage regimen for an adult human patient may be, for example, an oral dose of between 0.01 mg and 3000 mg of the active ingredient, preferably between 1 mg and 700 mg, e.g. 5 to 200 mg. In some embodiments, the daily dosage regimen is 1 mg, 5 mg, 10, mg, 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, or about any of the aforementioned numbers or a range bounded by any two of the aforementioned numbers. The dosage may be a single one or a series of two or more given in the course of one or more days, as is needed by the subject. In some embodiments, the compounds will be administered for a period of continuous therapy, for example for a week or more, or for months or years. Doses tailored for particular types of cancers or particular patients can be selected based, in part, on the GI₅₀, TGI, and LC₅₀ values determined or predicted for the particular type of cancer. Particularly preferred formulations for oral dosage include tablet or solutions, particularly solutions compatible with IV administration or solutions compatible with oral administration/use.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from EDso or IDso values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive conditions.

Dosage amount and interval may be adjusted individually to provide plasma levels of the active moiety which are sufficient to maintain the modulating effects, or minimal effective concentration (MEC). For example, therapeutic dosages may result in plasma levels of 0.05 μg/mL, 0.1 μg/mL, 0.5 μg/mL, 1 μg/mL, 5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, a range bounded by any two of the aforementioned numbers, or about any of the aforementioned numbers and ranges. In some embodiments, the therapeutic dose is sufficient to establish plasma levels in the range of about 0.1 μg/mL to about 10 μg/mL. In other embodiments, the therapeutic dose is sufficient to establish plasma levels in the range of 1 μg/mL to 20 μg/mL. The MEC may vary for each compound but can be estimated from in vitro or ex vivo data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

As described herein, the methods of the embodiments also include the use of a compound or compounds as described herein together with one or more additional therapeutic agents for the treatment of disease conditions. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially (e.g., in separate solution, emulsion, suspension, tablets, pills or capsules) or by different injections in separate syringes. In general, during alternation therapy, an effective dosage of each active ingredient is administered sequentially (e.g., serially), whereas in simultaneous therapy, effective dosages of two or more active ingredients are administered together. Various sequences of intermittent combination therapy may also be used.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 0-72 hrs, includes 1, 2, 3, 4, 5, 6, 7 hrs, etc., as well as 1, 2, 3, 4, 5, 6, 7 minutes, etc., and so forth. Reference to a range of 0-72 hrs, includes 1, 2, 3, 4, 5, 6, 7 hrs, etc., as well as 1, 2, 3, 4, 5, 6, 7 minutes, etc., and so forth. Reference to a range of doses, such as 0.1-1 μg/kg, 1-10 μg/kg, 10-25 μg/kg, 25-50 μg/kg, 50-100 μg/kg, 100-500 μg/kg, 500-1,000 μg/kg, 1-5 mg/kg, 5-10 mg/kg, 10-20 mg/kg, 20-50 mg/kg, 50-100 mg/kg, 100-250 mg/kg, 250-500 mg/kg, includes 0.11-0.9 μg/kg, 2-9 μg/kg, 11.5-24.5 μg/kg, 26-49 μg/kg, 55-90 μg/kg, 125-400 μg/kg, 750-800 μg/kg, 1.1-4.9 mg/kg, 6-9 mg/kg, 11.5-19.5 mg/kg, 21-49 mg/kg, 55-90 mg/kg, 125-200 mg/kg, 275.5-450.1 mg/kg, etc. A series of ranges, for example, 1-10 μg/kg, 10-25 μg/kg, 25-50 μg/kg, 50-100 μg/kg, 100-500 μg/kg, 500-1,000 μg/kg, 1-5 mg/kg, 5-10 mg/kg, 10-20 mg/kg, 20-50 mg/kg, 50-100 mg/kg, 100-250 mg/kg, 250-500 mg/kg, includes 1-25 μg/kg, 10-25 μg/kg, 25-100 μg/kg, 100-1,000 μg/kg, 1-10 mg/kg, 1-20 mg/kg etc.

### Co-Administration

As used herein, “co-administration” means concurrently or administering one substance followed by beginning the administration of a second substance within 24 hours, 20 hours, 16 hours, 12 hours, 8 hours, 4 hours, 1 hour, 30 minutes, 15 minutes, 5 minutes, 1 minute, a range bounded by any two of the aforementioned numbers, and/or about any of the aforementioned numbers. In some embodiments, co-administration is concurrent.

In some embodiments, one or more inhibitors of extracellular vesicle signaling are co-administered. In some embodiments, one or more inhibitors of extracellular vesicle signaling are co-administered with one or more chemotherapeutic agents. In other embodiments, the co-administration of one or more inhibitors of extracellular vesicle signaling accounts for the therapeutic benefit.

Some embodiments are directed to the use of companion diagnostics to identify an appropriate treatment for the patient. A companion diagnostic is an in vitro diagnostic test or device that provides information that is highly beneficial, or in some instances essential, for the safe and effective use of a corresponding therapeutic composition. Such tests or devices can identify patients likely to be at risk for adverse reactions as a result of treatment with a particular therapeutic composition. Such tests or devices can also monitor responsiveness to treatment (or estimate responsiveness to possible treatments). Such monitoring may include schedule, dose, discontinuation, or combinations of therapeutic compositions. In some embodiments, the inhibitor of extracellular vesicle signaling and/or other chemotherapeutic drug is selected by measuring a biomarker in the patient. The term biomarker includes, but is not limited to, genetic elements (e.g., presence/absence of a mutation and/or increase/decrease in expression level of a genetic element), proteins (e.g., presence/absence of a sequence/conformational mutation and/or increase/decrease in expression level of a protein), and cellular responses, such as cytotoxicity.

### CONCLUSION

Unless defined otherwise, all technical and scientific terms used herein have the same meaning as commonly understood by one of ordinary skill in the art to which the present disclosure pertains.

Any headings used herein are for organizational purposes only and are not meant to be used to limit the scope of the description or the claims.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A method for treating cancer, comprising receiving, at a subject in need thereof, a therapeutic dose of an inhibitor of extracellular vesicle signaling.

2. The method of claim 1, wherein the inhibitor of extracellular vesicle signaling is received by the subject as part of a pharmaceutical composition.

3. The method of claim 2, wherein the inhibitor of extracellular vesicle signaling comprises an inhibitor of neutral sphingomyelinase-2.

4. The method of claim 3, wherein treating cancer comprises treating pancreatic cancer.

5. The method of claim 2, wherein the inhibitor of extracellular vesicle signaling comprises GW4869.

6. The method of claim 1, further comprising receiving, at the subject in need thereof, a therapeutic dose or regimen of chemotherapy.

7. The method of claim 6, wherein the inhibitor of extracellular vesicle signaling comprises an inhibitor of neutral sphingomyelinase-2.

8. The method of claim 6, wherein the inhibitor of extracellular vesicle signaling comprises GW4869.

9. The method of claim 6, wherein treating cancer comprises treating one or more of colorectal cancer, pancreatic cancer, breast cancer, lung cancer, or prostate cancer.

10. A method for treating cancer, comprising administering a therapeutic dose of an inhibitor of extracellular vesicle signaling to a subject in need thereof.

11. The method of claim 10, wherein the inhibitor of extracellular vesicle signaling is administered to the subject as part of a pharmaceutical composition.

12. The method of claim 11, wherein the inhibitor of extracellular vesicle signaling comprises an inhibitor of neutral sphingomyelinase-2.

13. The method of claim 11, wherein the inhibitor of extracellular vesicle signaling comprises GW4869.

14. The method of claim 10, further comprising co-administering a therapeutic dose or regimen of chemotherapy to the subject in need thereof.

15. The method of claim 14, wherein the inhibitor of extracellular vesicle signaling comprises an inhibitor of neutral sphingomyelinase-2.

16. The method of claim 15, wherein treating cancer comprises treating pancreatic cancer.

17. The method of claim 14, wherein the inhibitor of extracellular vesicle signaling comprises GW4869.

18. The method of claim 17, wherein treating cancer comprises treating one or more of colorectal cancer, pancreatic cancer, breast cancer, lung cancer, or prostate cancer.

19. A method for treating pancreatic cancer, comprising:
administering a therapeutic dose of one or more inhibitors of extracellular vesicle signaling to a subject in need thereof, wherein at least one inhibitor of the one or more inhibitors of extracellular vesicle signaling comprises an inhibitor of neutral sphingomyelinase-2; and
co-administering a therapeutic dose or regimen of chemotherapy to the subject in need thereof.

20. The method of claim 19, wherein the inhibitor of extracellular vesicle signaling comprises GW4869.


SEQUENCE LISTING (10 sequences)

SEQ ID NO: 1 - DNA, 30 bp, Homo sapiens
ttggagctggtggcgtaggcagagtgcctt

SEQ ID NO: 2 - DNA, 30 bp, Homo sapiens; identical to SEQ ID NO: 1

SEQ ID NO: 3 - DNA, 30 bp, Homo sapiens; identical to SEQ ID NO: 1

SEQ ID NO: 4 - RNA, 22 nt, Homo sapiens
ccucugaaauucaguucuucag

SEQ ID NO: 5 - RNA, 22 nt, Homo sapiens
ugagaacugaauuccauggguu

SEQ ID NO: 6 - RNA, 22 nt, Homo sapiens
uagcuuaucagacugauguuga

SEQ ID NO: 7 - RNA, 23 nt, Homo sapiens
aacauucaacgcugucggugagu

SEQ ID NO: 8 - RNA, 23 nt, Homo sapiens
agcuacauugucugcuggguuuc

SEQ ID NO: 9 - RNA, 21 nt, Homo sapiens
agcuacaucuggcuacugggu

SEQ ID NO: 10 - RNA, 22 nt, Homo sapiens
uauugcacuugucccggccugu